(19) United States
(12) Patent Application Publication
(10) Pub. No.: US 2015/0166426 A1
(43) Pub. Date: Jun. 18, 2015

(54) PROCESS FOR OLIGOMERIZING TO MAXIMIZE NONENES FOR CRACKING TO PROPYLENE

(71) Applicant: UOP LLC, Des Plaines, IL (US)

(72) Inventors: David A. Wegerer, Lisle, IL (US); Kurt M. Vanden Bussche, Lake in the Hills, IL (US); Todd M. Kruse, Oak Park, IL (US)

(73) Assignee: UOP LLC, Des Plaines, IL (US)

(21) Appl. No.: 14/108,623

(22) Filed: Dec. 17, 2013

(51) Int. Cl. C07C 2/08; C07C 4/06

(52) U.S. Cl. CPC C07C 2/08; C07C 4/06

(57) ABSTRACT

To bias an oligomerization reaction toward C₉ olefin production, C₅ olefins are split and fed to a C₄ olefin feed stream at a downstream location, so the C₄ olefins are in stoichiometric excess over the C₅ olefins. The result is greater oligomerization to C₉ olefins. C₉ olefins fed to an FCC unit have a carbon number divisible by three and thus produces a greater proportion of propylene.

### BACKGROUND

The field of the invention is the oligomerization of light olefins to heavier oligomers that can be cracked to propylene.

To maximize propylene produced by an FCC unit, refiners may contemplate oligomerizing FCC olefins to make heavier oligomers and recycling heavier oligomers to the FCC unit. Often an oligomerization unit is employed to oligomerize C₄ olefins to make olefins with eight carbons. This eight carbon product is then sent back to an FCC unit to be re-cracked to make more propylene. However, olefins with eight carbons are not the ideal to be sent back to an FCC unit to make more propylene because olefins with other carbon numbers other than three will necessarily be made.

It would be preferable to make olefins with nine carbons which can be more easily re-cracked to make propylene. A product stream of C₅ olefins is typically available to add to the C₄ olefin stream to try to make olefins with nine carbons, but adding this stream to the C₄ olefin feed can simply lead to the formation of too many olefins with ten carbons instead of nine carbons.

A process is needed that can maximize the formation of olefins with nine carbons from a mixed stream of C₄ olefins and C₅ olefins.

This process is needed to provide more olefins with nine carbons that can be sent to an FCC unit to produce more propylene than could have otherwise been achieved with more olefins with eight carbons.

### SUMMARY

A process is described for feeding C₅ olefins to the reactor in a series of side ports. The process provides an excess of C₄ olefins at each pentene feed point which then favors the formation of olefins with nine carbons. The net product maximizes the amount of olefins with nine carbons produced.

An embodiment is a process for making olefins comprising feeding a first feed stream comprising C₄ olefins to an oligomerization reactor having an inlet end and an outlet end; feeding a second feed stream comprising C₅ olefins to the oligomerization reactor at a first inlet; feeding a third feed stream comprising C₅ olefins to an oligomerization reactor at a second inlet that is downstream of the first inlet; and oligomerizing the C₄ olefins and the C₅ olefins over an oligomerization catalyst to produce an oligomerate stream comprising C₉ olefins.

An object of the invention is to enable an oligomerization unit to make more C₉ olefin compounds which can be cracked to propylene in an FCC unit.

### BRIEF DESCRIPTION OF THE DRAWINGS

FIG. 1 is a schematic drawing of the present invention.

FIG. 2 is a plot of C₈-C₁₁ olefin selectivity versus normal butene conversion.

FIG. 3 is a plot of C₁₂+ olefin selectivity versus normal butene conversion.

FIG. 4 is a plot of conversion versus total butene conversion.

FIG. 5 is a plot of C₃ olefin yield versus VGO conversion.

### DEFINITIONS

As used herein, the term “stream” can include various hydrocarbon molecules and other substances. Moreover, the term “stream comprising Cₓ hydrocarbons” or “stream comprising Cₓ olefins” can include a stream comprising hydrocarbon or olefin molecules, respectively, with “x” number of carbon atoms, suitably a stream with a majority of hydrocarbons or olefins, respectively, with “x” number of carbon atoms and preferably a stream with at least 75 wt % hydrocarbons or olefin molecules, respectively, with “x” number of carbon atoms. Moreover, the term “stream comprising Cₓ+ hydrocarbons” or “stream comprising Cₓ+ olefins” can include a stream comprising a majority of hydrocarbon or olefin molecules, respectively, with more than or equal to “x” carbon atoms and suitably less than 10 wt % and preferably less than 1 wt % hydrocarbon or olefin molecules, respectively, with x−1 carbon atoms. Lastly, the term “Cₓ-stream” can include a stream comprising a majority of hydrocarbon or olefin molecules, respectively, with less than or equal to “x” carbon atoms and suitably less than 10 wt % and preferably less than 1 wt % hydrocarbon or olefin molecules, respectively, with x+1 carbon atoms.

As used herein, the term “zone” can refer to an area including one or more equipment items and/or one or more sub-zones. Equipment items can include one or more reactors or reactor vessels, heaters, exchangers, pipes, pumps, compressors, controllers and columns. Additionally, an equipment item, such as a reactor, dryer, or vessel, can further include one or more zones or sub-zones.

As used herein, the term “substantially” can mean an amount of at least generally about 70%, preferably about 80%, and optimally about 90%, by weight, of a compound or class of compounds in a stream.

As used herein, the term “gasoline” can include hydrocarbons having a boiling point temperature in the range of about 25 to about 200° C. at atmospheric pressure.

As used herein, the term “diesel” or “distillate” can include hydrocarbons having a boiling point temperature in the range of about 150° to about 400° C. and preferably about 200° to about 400° C.

As used herein, the term “vacuum gas oil” (VGO) can include hydrocarbons having a boiling temperature in the range of from 343° to 552° C.

As used herein, the term “vapor” can mean a gas or a dispersion that may include or consist of one or more hydrocarbons.

As used herein, the term “overhead stream” can mean a stream withdrawn at or near a top of a vessel, such as a column.

As used herein, the term “bottom stream” can mean a stream withdrawn at or near a bottom of a vessel, such as a column.

As depicted, process flow lines in the figures can be referred to interchangeably as, e.g., lines, pipes, feeds, gases, products, discharges, parts, portions, or streams.

As used herein, “bypassing” with respect to a vessel or zone means that a stream does not pass through the zone or vessel bypassed although it may pass through a vessel or zone that is not designated as bypassed.

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without undergoing a compositional change due to physical fractionation or chemical conversion.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottom stream back to the bottom of the column. Feeds to the columns may be preheated. The top pressure is the pressure of the overhead vapor at the outlet of the column. The bottom temperature is the liquid bottom outlet temperature. Overhead lines and bottom lines refer to the net lines from the column downstream of the reflux or reboil to the column.

As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, “taking a stream from” means that some or all of the original stream is taken.

### DETAILED DESCRIPTION

The present process feeds C₅ olefins to an oligomerization reactor in a side port which is downstream of an upstream inlet for C₄ olefins to the reactor. This process allows an excess of C₄ olefin at each pentene feed point which then favors the formation of C₉ olefins. Typically, an FCC feed produces a 2:1 ratio of C₄ olefins to C₅ olefins the process can be employed consistently to make as much C₉ olefins as possible. Since the carbon number of C₉ olefins is divisible by three, cracking is more likely to produce cracked products with high selectivity to propylene.

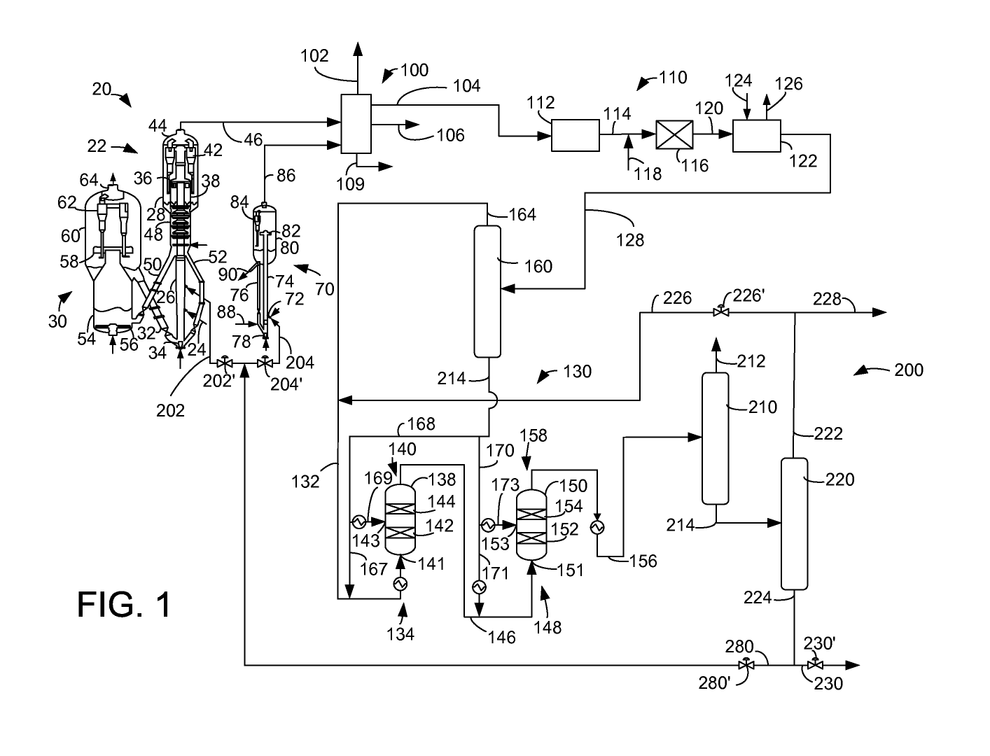

The process may be described with reference to five components shown in FIG. 1: a fluid catalytic cracking (FCC) zone 20, an FCC recovery zone 100, a purification zone 110, an oligomerization zone 130, and an oligomerization recovery zone 200. Many configurations of the present invention are possible, but specific embodiments are presented herein by way of example. All other possible embodiments for carrying out the present invention are considered within the scope of the present invention.

The FCC zone 20 may comprise a first FCC reactor 22, a regenerator vessel 30, and an optional second FCC reactor 70.

A conventional FCC feedstock and higher boiling hydrocarbon feedstock are a suitable FCC hydrocarbon feed 24 to the first FCC reactor. The most common of such conventional feedstocks is a VGO. Higher boiling hydrocarbon feedstocks to which this invention may be applied include heavy bottom from crude oil, heavy bitumen crude oil, shale oil, tar sand extract, deasphalted residue, products from coal liquefaction, atmospheric and vacuum reduced crudes and mixtures thereof. The FCC feed 24 may include an FCC recycle stream from an FCC recycle line 280 to be described later.

The first FCC reactor 22 may include a first reactor riser 26 and a first reactor vessel 28. A regenerator catalyst pipe 32 delivers regenerated catalyst from the regenerator vessel 30 to the reactor riser 26. A fluidization medium such as steam from a distributor 34 urges a stream of regenerated catalyst upwardly through the first reactor riser 26. At least one feed distributor injects the first hydrocarbon feed in a first hydrocarbon feed line 24, preferably with an inert atomizing gas such as steam, across the flowing stream of catalyst particles to distribute hydrocarbon feed to the first reactor riser 26. Upon contacting the hydrocarbon feed with catalyst in the first reactor riser 26 the heavier hydrocarbon feed cracks to produce lighter gaseous cracked products while coke is deposited on the catalyst particles to produce spent catalyst.

The resulting mixture of gaseous product hydrocarbons and spent catalyst continues upwardly through the first reactor riser 26 and are received in the first reactor vessel 28 in which the spent catalyst and gaseous product are separated. Disengaging arms discharge the mixture of gas and catalyst from a top of the first reactor riser 26 through outlet ports 36 into a disengaging vessel 38 that effects partial separation of gases from the catalyst. A transport conduit carries the hydrocarbon vapors, stripping media and entrained catalyst to one or more cyclones 42 in the first reactor vessel 28 which separates spent catalyst from the hydrocarbon gaseous product stream. Gas conduits deliver separated hydrocarbon cracked gaseous streams from the cyclones 42 to a collection plenum 44 for passage of a cracked product stream to a first cracked product line 46 via an outlet nozzle and eventually into the FCC recovery zone 100 for product recovery.

Diplegs discharge catalyst from the cyclones 42 into a lower bed in the first reactor vessel 28. The catalyst with adsorbed or entrained hydrocarbons may eventually pass from the lower bed into a stripping section 48 across ports defined in a wall of the disengaging vessel 38. Catalyst separated in the disengaging vessel 38 may pass directly into the stripping section 48 via a bed. A fluidizing distributor delivers inert fluidizing gas, typically steam, to the stripping section 48. The stripping section 48 contains baffles or other equipment to promote contacting between a stripping gas and the catalyst. The stripped spent catalyst leaves the stripping section 48 of the disengaging vessel 38 of the first reactor vessel 28 stripped of hydrocarbons. A first portion of the spent catalyst, preferably stripped, leaves the disengaging vessel 38 of the first reactor vessel 28 through a spent catalyst conduit 50 and passes into the regenerator vessel 30. A second portion of the spent catalyst may be recirculated in recycle conduit 52 from the disengaging vessel 38 back to a base of the first riser 26 at a rate regulated by a slide valve to recontact the feed without undergoing regeneration.

The first riser 26 can operate at any suitable temperature, and typically operates at a temperature of about 150° to about 580° C. at the riser outlet 36. The pressure of the first riser is from about 69 to about 517 kPa (gauge) (10 to 75 psig) but typically less than about 275 kPa (gauge) (40 psig). The catalyst-to-oil ratio, based on the weight of catalyst and feed hydrocarbons entering the riser, may range up to 30:1 but is typically between about 4:1 and about 25:1. Steam may be passed into the first reactor riser 26 and first reactor vessel 28 at a rate between about 2 and about 7 wt % for maximum gasoline production and about 10 to about 30 wt % for maximum light olefin production. The average residence time of catalyst in the riser may be less than about 5 seconds.

The catalyst in the first reactor 22 can be a single catalyst or a mixture of different catalysts. Usually, the catalyst includes two catalysts, namely a first FCC catalyst, and a second FCC catalyst. Such a catalyst mixture is disclosed in, e.g., U.S. Pat. No. 7,312,370 B2. Generally, the first FCC catalyst may include any of the well-known catalysts that are used in the art of FCC. Preferably, the first FCC catalyst includes a large pore zeolite, such as a Y-type zeolite, an active alumina material, a binder material, including either silica or alumina, and an inert filler such as kaolin.

Typically, the zeolites appropriate for the first FCC catalyst have a large average pore size, usually with openings of greater than about 0.7 nm in effective diameter defined by greater than about 10, and typically about 12, member rings. Suitable large pore zeolite components may include synthetic zeolites such as X and Y zeolites, mordenite and faujasite. A portion of the first FCC catalyst, such as the zeolite portion, can have any suitable amount of a rare earth metal or rare earth metal oxide.

The second FCC catalyst may include a medium or smaller pore zeolite catalyst, such as exemplified by at least one of ZSM-5, ZSM-11, ZSM-12, ZSM-23, ZSM-35, ZSM-38, ZSM-48, and other similar materials. Other suitable medium or smaller pore zeolites include ferrierite, and erionite. Preferably, the second component has the medium or smaller pore zeolite dispersed on a matrix including a binder material such as silica or alumina and an inert filler material such as kaolin. These catalysts may have a crystalline zeolite content of about 10 to about 50 wt % or more, and a matrix material content of about 50 to about 90 wt %. Catalysts containing at least about 40 wt % crystalline zeolite material are typical, and those with greater crystalline zeolite content may be used. Generally, medium and smaller pore zeolites are characterized by having an effective pore opening diameter of less than or equal to about 0.7 nm and rings of about 10 or fewer members. Preferably, the second FCC catalyst component is an MFI zeolite having a silicon-to-aluminum ratio greater than about 15. In one exemplary embodiment, the silicon-to-aluminum ratio can be about 15 to about 35.

The total catalyst mixture in the first reactor 22 may contain about 1 to about 25 wt % of the second FCC catalyst, including a medium to small pore crystalline zeolite, with greater than or equal to about 7 wt % of the second FCC catalyst being preferred. When the second FCC catalyst contains about 40 wt % crystalline zeolite with the balance being a binder material, an inert filler, such as kaolin, and optionally an active alumina component, the catalyst mixture may contain about 0.4 to about 10 wt % of the medium to small pore crystalline zeolite with a preferred content of at least about 2.8 wt %. The first FCC catalyst may comprise the balance of the catalyst composition. The high concentration of the medium or smaller pore zeolite as the second FCC catalyst of the catalyst mixture can improve selectivity to light olefins. In one exemplary embodiment, the second FCC catalyst can be a ZSM-5 zeolite and the catalyst mixture can include about 0.4 to about 10 wt % ZSM-5 zeolite excluding any other components, such as binder and/or filler.

The regenerator vessel 30 is in downstream communication with the first reactor vessel 28. In the regenerator vessel 30, coke is combusted from the portion of spent catalyst delivered to the regenerator vessel 30 by contact with an oxygen-containing gas such as air to regenerate the catalyst. The spent catalyst conduit 50 feeds spent catalyst to the regenerator vessel 30. The spent catalyst from the first reactor vessel 28 usually contains carbon in an amount of from 0.2 to 7 wt %, which is present in the form of coke. An oxygen-containing combustion gas, typically air, enters the lower chamber 54 of the regenerator vessel 30 through a conduit and is distributed by a distributor 56. As the combustion gas enters the lower chamber 54, it contacts spent catalyst entering from spent catalyst conduit 50 and lifts the catalyst at a superficial velocity of combustion gas in the lower chamber 54 of perhaps at least 1.1 m/s (3.5 ft/s) under fast fluidized flow conditions. In an embodiment, the lower chamber 54 may have a catalyst density of from 48 to 320 kg/m³ (3 to 20 lb/ft³) and a superficial gas velocity of 1.1 to 2.2 m/s (3.5 to 7 ft/s). The oxygen in the combustion gas contacts the spent catalyst and combusts carbonaceous deposits from the catalyst to at least partially regenerate the catalyst and generate flue gas.

The mixture of catalyst and combustion gas in the lower chamber 54 ascends through a frustoconical transition section to the transport, riser section of the lower chamber 54. The mixture of catalyst particles and flue gas is discharged from an upper portion of the riser section into the upper chamber 60. Substantially completely or partially regenerated catalyst may exit the top of the transport, riser section. Discharge is effected through a disengaging device 58 that separates a majority of the regenerated catalyst from the flue gas. The catalyst and gas exit downwardly from the disengaging device 58. The sudden loss of momentum and downward flow reversal cause a majority of the heavier catalyst to fall to the dense catalyst bed and the lighter flue gas and a minor portion of the catalyst still entrained therein to ascend upwardly in the upper chamber 60. Cyclones 62 further separate catalyst from ascending gas and deposits catalyst through dip legs into a dense catalyst bed. Flue gas exits the cyclones 62 through a gas conduit and collects in a plenum 64 for passage to an outlet nozzle of regenerator vessel 30. Catalyst densities in the dense catalyst bed are typically kept within a range of from about 640 to about 960 kg/m³ (40 to 60 lb/ft³).

The regenerator vessel 30 typically has a temperature of about 594° to about 704° C. (1100° to 1300° F.) in the lower chamber 54 and about 649° to about 760° C. (1200° to 1400° F.) in the upper chamber 60. Regenerated catalyst from dense catalyst bed is transported through regenerated catalyst pipe 32 from the regenerator vessel 30 back to the first reactor riser 26 through the control valve where it again contacts the first feed in line 24 as the FCC process continues. The first cracked product stream in the first cracked product line 46 from the first reactor 22, relatively free of catalyst particles and including the stripping fluid, exit the first reactor vessel 28 through an outlet nozzle. The first cracked products stream in the line 46 may be subjected to additional treatment to remove fine catalyst particles or to further prepare the stream prior to fractionation. The line 46 transfers the first cracked products stream to the FCC recovery zone 100, which is in downstream communication with the FCC zone 20. The FCC recovery zone 100 typically includes a main fractionation column and a gas recovery section. The FCC recovery zone can include many fractionation columns and other separation equipment.

The FCC recovery zone 100 can recover a propylene product stream in propylene line 102, a light olefin stream in light olefin line 104, a gasoline stream in gasoline line 106 and an LCO stream in LCO line 109 among others from the cracked product stream in the first cracked product line 46. The light olefin stream in light olefin line 104 comprises an oligomerization feed stream having C₄ hydrocarbons including C₄ olefins and perhaps having C₅ hydrocarbons including C₅ olefins.

An FCC recycle stream in an recycle line 280 delivers an FCC recycle stream to the FCC zone 20. The FCC recycle stream is directed into a first FCC recycle line 202 with the control valve 202′ thereon opened. In an aspect, the FCC recycle stream may be directed into an optional second FCC recycle line 204 with the control valve 204′ thereon opened. The first FCC recycle line 202 delivers the first FCC recycle stream to the first FCC reactor 22 in an aspect to the riser 26 at an elevation above the first hydrocarbon feed in line 24. The second FCC recycle line 204 delivers the second FCC recycle stream to the second FCC reactor 70. Typically, both control valves 202′ and 204′ will not be opened at the same time, so the FCC recycle stream goes through only one of the first FCC recycle line 202 and the second FCC recycle line 204. However, feed through both is contemplated.

The second FCC recycle stream may be fed to the second FCC reactor 70 in the second FCC recycle line 204 via feed distributor 72. The second FCC reactor 70 may include a second riser 74. The second FCC recycle stream is contacted with catalyst delivered to the second riser 74 by a catalyst return pipe 76 to produce cracked upgraded products. The catalyst may be fluidized by inert gas such as steam from distributor 78. Generally, the second FCC reactor 70 may operate under conditions to convert the second FCC recycle stream to second cracked products such as ethylene and propylene. A second reactor vessel 80 is in downstream communication with the second riser 74 for receiving second cracked products and catalyst from the second riser. The mixture of gaseous, second cracked product hydrocarbons and catalyst continues upwardly through the second reactor riser 74 and is received in the second reactor vessel 80 in which the catalyst and gaseous, second cracked products are separated. A pair of disengaging arms may tangentially and horizontally discharge the mixture of gas and catalyst from a top of the second reactor riser 74 through one or more outlet ports 82 (only one is shown) into the second reactor vessel 80 that effects partial separation of gases from the catalyst. The catalyst can drop to a dense catalyst bed within the second reactor vessel 80. Cyclones 84 in the second reactor vessel 80 may further separate catalyst from second cracked products. Afterwards, a second cracked product stream can be removed from the second reactor 84 through an outlet in a second cracked product line 86 in downstream communication with the second reactor riser 74. The second cracked product stream in line 86 is fed to the FCC recovery zone 100, preferably separately from the first cracked products to undergo separation and recovery of ethylene and propylene. Separated catalyst may be recycled via a recycle catalyst pipe 76 from the second reactor vessel 80 regulated by a control valve back to the second reactor riser 74 to be contacted with the second FCC recycle stream.

In some embodiments, the second FCC reactor 70 can contain a mixture of the first and second FCC catalysts as described above for the first FCC reactor 22. In one preferred embodiment, the second FCC reactor 70 can contain less than about 20 wt %, preferably less than about 5 wt % of the first FCC catalyst and at least 20 wt % of the second FCC catalyst. In another preferred embodiment, the second FCC reactor 70 can contain only the second FCC catalyst, preferably a ZSM-5 zeolite.

The second FCC reactor 70 is in downstream communication with the regenerator vessel 30 and receives regenerated catalyst therefrom in line 88. In an embodiment, the first FCC reactor 22 and the second FCC reactor 70 both share the same regenerator vessel 30. Line 90 carries spent catalyst from the second reactor vessel 80 to the lower chamber 54 of the regenerator vessel 30. The catalyst regenerator is in downstream communication with the second FCC reactor 70 via line 90.

The same catalyst composition may be used in both reactors 22, 70. However, if a higher proportion of the second FCC catalyst of small to medium pore zeolite is desired in the second FCC reactor 70 than the first FCC catalyst of large pore zeolite, replacement catalyst added to the second FCC reactor 70 may comprise a higher proportion of the second FCC catalyst. Because the second FCC catalyst does not lose activity as quickly as the first FCC catalyst, less of the second catalyst inventory must be forwarded to the catalyst regenerator 30 in line 90 from the second reactor vessel 80, but more catalyst inventory may be recycled to the riser 74 in return conduit 76 without regeneration to maintain a high level of the second FCC catalyst in the second reactor 70.

The second reactor riser 74 can operate in any suitable condition, such as a temperature of about 425° to about 705° C., preferably a temperature of about 550° to about 600° C., and a pressure of about 140 to about 400 kPa, preferably a pressure of about 170 to about 250 kPa. Typically, the residence time of the second reactor riser 74 can be less than about 3 seconds and preferably is than about 1 second. Exemplary risers and operating conditions are disclosed in, e.g., U.S. Pat. No. 7,491,315 and U.S. Pat. No. 7,261,807.

Before cracked products can be fed to the oligomerization zone 130, the light olefin stream in light olefin line 104 may require purification. Many impurities in the light olefin stream in light olefin line 104 can poison an oligomerization catalyst. Carbon dioxide and ammonia can attack acid sites on the catalyst. Sulfur containing compounds, oxygenates, and nitriles can harm oligomerization catalyst. Acetylenes and diolefins can polymerize and produce gums on the catalyst or equipment. Consequently, the light olefin stream which comprises the oligomerization feed stream in light olefin line 104 may be purified in an optional purification zone 110.

The light olefin stream in light olefin line 104 may be introduced into an optional mercaptan extraction unit 112 to remove mercaptans to lower concentrations. In the mercaptan extraction unit 112, the light olefin feed may be prewashed in an optional prewash vessel containing aqueous alkali to convert any hydrogen sulfide to sulfide salt which is soluble in the aqueous alkaline stream. The light olefin stream, now depleted of any hydrogen sulfide, is contacted with a more concentrated aqueous alkali stream in an extractor vessel. Mercaptans in the light olefin stream react with the alkali to yield mercaptides. An extracted light olefin stream lean in mercaptans passes overhead from the extraction column and may be mixed with a solvent that removes COS in route to an optional COS solvent settler. COS is removed with the solvent from the bottom of the settler, while the overhead light olefin stream may be fed to an optional water wash vessel to remove remaining alkali and produce a sulfur depleted light olefin stream in line 114. The mercaptide rich alkali from the extractor vessel receives an injection of air and a catalyst such as phthalocyanine as it passes from the extractor vessel to an oxidation vessel for regeneration. Oxidizing the mercaptides to disulfides using a catalyst regenerates the alkaline solution. A disulfide separator receives the disulfide rich alkaline from the oxidation vessel. The disulfide separator vents excess air and decants disulfides from the alkaline solution before the regenerated alkaline is drained, washed with oil to remove remaining disulfides and returned to the extractor vessel. Further removal of disulfides from the regenerated alkaline stream is also contemplated. The disulfides may be run through a sand filter and removed from the process. For more information on mercaptan extraction, reference may be made to U.S. Pat. No. 7,326,333.

In order to prevent polymerization and gumming in the oligomerization reactor that can inhibit equipment and catalyst performance, it is desired to minimize diolefins and acetylenes in the light olefin feed in line 114. Diolefin conversion to monoolefin hydrocarbons may be accomplished by selectively hydrogenating the sulfur depleted stream with a conventional selective hydrogenation reactor 116. Hydrogen may be added to the purified light olefin stream in line 118.

The selective hydrogenation catalyst can comprise an alumina support material preferably having a total surface area greater than 150 m²/g, with most of the total pore volume of the catalyst provided by pores with average diameters of greater than 600 angstroms, and containing surface deposits of about 1.0 to 25.0 wt % nickel and about 0.1 to 1.0 wt % sulfur such as disclosed in U.S. Pat. No. 4,695,560. Spheres having a diameter between about 0.4 and 6.4 mm ( 1/64 and ¼ inch) can be made by oil dropping a gelled alumina sol. The alumina sol may be formed by digesting aluminum metal with an aqueous solution of approximately 12 wt % hydrogen chloride to produce an aluminum chloride sol. The nickel component may be added to the catalyst during the sphere formation or by immersing calcined alumina spheres in an aqueous solution of a nickel compound followed by drying, calcining, purging and reducing. The nickel containing alumina spheres may then be sulfided. A palladium catalyst may also be used as the selective hydrogenation catalyst.

The selective hydrogenation process is normally performed at relatively mild hydrogenation conditions. These conditions will normally result in the hydrocarbons being present as liquid phase materials. The reactants will normally be maintained under the minimum pressure sufficient to maintain the reactants as liquid phase hydrocarbons which allow the hydrogen to dissolve into the light olefin feed. A broad range of suitable operating pressures therefore extends from about 276 (40 psig) to about 5516 kPa gauge (800 psig). A relatively moderate temperature between about 25° C. (77° F.) and about 350° C. (662° F.) should be employed. The liquid hourly space velocity of the reactants through the selective hydrogenation catalyst should be above 1.0 hr⁻¹. Preferably, it is between 5.0 and 35.0 hr⁻¹. The molar ratio of hydrogen to diolefinic hydrocarbons may be maintained between 1.5:1 and 2:1. The hydrogenation reactor is preferably a cylindrical fixed bed of catalyst through which the reactants move in a vertical direction.

A purified light olefin stream depleted of sulfur containing compounds, diolefins and acetylenes exits the selective hydrogenation reactor 116 in line 120. The optionally sulfur and diolefin depleted light olefin stream in line 120 may be introduced into an optional nitrile removal unit such as a water wash unit 122 to reduce the concentration of oxygenates and nitriles in the light olefin stream in line 120. Water is introduced to the water wash unit in line 124. An oxygenate and nitrile-rich aqueous stream in line 126 leaves the water wash unit 122 and may be further processed. A drier may follow the water wash unit 122. Other nitrile removal units (NRU) may be used in place of the water wash. A NRU usually consists of a group of regenerable beds that adsorb the nitriles and other nitrogen components from the purified light olefins stream. Examples of nitrogen removal units can be found in U.S. Pat. No. 4,831,206, U.S. Pat. No. 5,120,881 and U.S. Pat. No. 5,271,835.

A purified light olefin oligomerization feed stream perhaps depleted of sulfur containing compounds, diolefins and/or oxygenates and nitriles is provided in oligomerization feed stream line 128. The light olefin oligomerization feed stream in line 128 may be obtained from the cracked product stream in lines 46 and/or 86, so it may be in downstream communication with the FCC zone 20. The oligomerization feed stream need not be obtained from a cracked FCC product stream but may come from another source. The selective hydrogenation reactor 116 is in upstream communication with the oligomerization feed stream line 128. The oligomerization feed stream may comprise C₄ hydrocarbons such as C₄ olefins, i.e., butenes, and butanes. C₄ olefins include normal butenes and isobutene. The oligomerization feed stream in oligomerization feed stream line 128 may comprise C₅ hydrocarbons such as C₅ olefins, i.e., pentenes, and pentanes. C₅ olefins include normal pentenes and isopentenes. Typically, the oligomerization feed stream will comprise about 20 to about 80 wt % olefins and suitably about 40 to about 75 wt % olefins. In an aspect, about 55 to about 75 wt % of the olefins may be C₄ olefins and about 25 to about 45 wt % of the olefins may be C₅ olefins. Up to 10 wt %, suitably 20 wt %, typically 25 wt % and most typically 30 wt % of the oligomerization feed may be C₅ olefins.

An aspect of the present process is to split C₄ olefins from the C₅ olefins prior to feeding them to the oligomerization zone 130. Consequently, the oligomerization feed stream in the oligomerization feed stream line 128 is fed to a debutanizer column 160 upstream of the oligomerization zone 130. The debutanizer column 160 may be in downstream communication with the FCC zone 20 and upstream of the oligomerization zone 130. The debutanizer column fractionates the oligomerization feed stream into an overhead stream comprising C₄− hydrocarbons and bottoms stream comprising C₅+ hydrocarbons. The debutanizer column may be operated at a top pressure of about 1034 to about 1724 kPa (gauge) (150 to 250 psig) and a bottom temperature of about 149° to about 204° C. (300° to 400° F.). The pressure should be maintained as low as possible to maintain a reboiler temperature as low as possible while still allowing complete condensation with typical cooling utilities without the need for refrigeration. The overhead stream in line 164 from the debutanizer comprises C₄ olefin feed which can be sent to an upstream inlet of the oligomerization zone 130. The bottoms stream in line 214 comprising C₅ olefins may be split between a first stream comprising C₅ olefins in a first pentene line 168 and a second stream comprising C₅ olefins in second pentene line 170 for delivering C₅ olefins to different locations in the oligomerization zone 130. At least about 40 wt % of the stream comprising C₅ olefins in the bottoms line 214 may be normal pentene. In an aspect, no more than about 70 wt % of the stream comprising C₅ olefins in the bottoms line 213 may be normal pentene.

The overhead stream in overhead line 164 feeds the C₄ olefin feed stream to an oligomerization zone 130 which may be in downstream communication with the FCC recovery zone 100 and the debutanizer column 160. The C₄ olefin feed stream in overhead line 164 may be mixed with an oligomerate recycle stream in line 226 prior to entering the oligomerization zone 130 to provide a first feed stream of C₄ olefins in a first feed conduit 132.

The oligomerization zone 130 comprises an oligomerization reactor 138. The oligomerization reactor may be preceded by an optional guard bed for removing catalyst poisons that is not shown. The oligomerization reactor 138 is in downstream communication with the first feed conduit 132. The oligomerization reactor 138 contains an oligomerization catalyst.

The first feed stream of C₄ olefins in the first feed conduit 132 may comprise about 15 to about 85 wt % C₄ olefins and suitably about 40 to about 70 wt % C₄ olefins. The first feed stream to the oligomerization zone 130 in the first feed conduit 132 may comprise at least about 10 wt % C₄ olefin and preferably no more than about 1 wt % hexene. In a further aspect, the first feed stream may comprise no more than about 0.1 wt % hexene and no more than about 0.1 wt % propylene. At least about 40 wt % of the C₄ olefin in the first feed stream may be normal butene. In an aspect, it may be that no more than about 70 wt % of the first feed stream is normal butene.

The first stream comprising C₅ olefins in the first pentene line 168 may be split into a second feed stream in a second feed conduit 167 and a third feed stream in a third feed conduit 169. The second stream comprising C₅ olefins in the second pentene line 170 may be split into a fourth feed stream in a fourth feed conduit 171 and a fifth feed stream in a fifth feed conduit 173. The division of the streams comprising C₅ olefins is designed to reduce the volume of these C₅ olefins streams in aliquot proportions.

The first feed stream of C₄ olefins in the first feed conduit 132 may be fed to a first inlet 141 to the oligomerization reactor 138. The first inlet 141 may be provided at an inlet end 134 of the oligomerization reactor 138 and the oligomerization zone 130. The second feed stream of C₅ olefins in the second feed conduit 167 may also be fed to the first inlet 141 of the first oligomerization reactor 138. The first feed stream and the second feed stream may be fed to the oligomerization reactor 138 together through the first feed conduit 132 to the first inlet 141 or in separate conduits or through separate inlets. The first feed stream may be heat exchanged before entering the oligomerization reactor 138. The oligomerization reactor 138 may contain a first catalyst bed 142 of oligomerization catalyst. The oligomerization reactor 138 may be an upflow reactor to provide a uniform feed front through the catalyst bed, but other flow arrangements are contemplated. In an aspect, the oligomerization reactor 138 may contain an additional bed or beds 144 of oligomerization catalyst.

C₄ olefins in the first feed stream oligomerize over the oligomerization catalyst to provide an oligomerate comprising C₄ olefin dimers and trimers. C₅ olefins that may be present in the first feed stream oligomerize over the oligomerization catalyst to provide an oligomerate comprising C₅ olefin dimers and trimers and co-oligomerize with C₄ olefins to make C₉ olefins.

The third feed stream of C₅ olefins in a third feed conduit 169 is fed to a second inlet 143 to the oligomerization reactor 138. The second inlet may be arranged to provide feed to the bed 144 or to an interbed location between beds 142 and an additional bed 144. However, the second inlet 143 is downstream of the first inlet 141 relative to feed flow through the oligomerization reactor 138 and the oligomerization zone 130. The third feed stream of C₅ olefins may serve as a quench for the effluent from the first bed 142 to avoid excessive temperature rise. A cooler may be on the third feed conduit 169 to facilitate quenching. Additional oligomerization occurs across bed 144. Oligomerized product, in an oligomerate stream, exits the first oligomerization reactor 138 in an effluent line 146. The effluent line exits the first oligomerization reactor 138 at a first outlet end 140 of the oligomerization reactor 138.

A stoichiometric surplus of C₄ olefins to C₅ olefins should be maintained in the feed to the first bed 142 and to the additional bed 144 to promote co-oligomerization of C₄ olefins with C₅ olefins to form nonene oligomers. The second feed stream of C₅ olefins in the second feed line 167 and said third feed stream of C₅ olefins in the third feed line 169 should have smaller mass and molar flow rates than the first feed stream of C₄ olefins in the first feed conduit 132. For example, the weight ratio of C₄ olefins to C₅ olefins in the reactor should be between about 1.5 and about 3.0 and preferably between about 1.7 and about 2.5 at the first inlet 141 and the second inlet 143 through which a C₅ olefin feed stream is added to the oligomerization reactor 138. Consequently, nonene production is maximized due to the stoichiometric excess of C₄ olefins over C₅ olefins at the feed inlets in the oligomerization reactor 138.

In an aspect, the oligomerization reactor zone may include one or more additional oligomerization reactors 150. The oligomerization effluent may be heated and fed to the optional additional oligomerization reactor 150. It is contemplated that the first oligomerization reactor 138 and the additional oligomerization reactor 150 may be operated in a swing bed fashion to take one reactor offline for maintenance or catalyst regeneration or replacement while the other reactor stays online. In an aspect, the additional oligomerization reactor 150 may contain a first bed 152 of oligomerization catalyst. The additional oligomerization reactor 150 may also be an upflow reactor to provide a uniform feed front through the catalyst bed, but other flow arrangements are contemplated. In an aspect, the additional oligomerization reactor 150 may contain an additional bed or beds 154 of oligomerization catalyst. It is also contemplated that all of the catalyst beds 142, 144, 152, and 154 may be contained in a single oligomerization reactor.

The oligomerate stream in effluent line 146 comprising unreacted C₄ olefins may be fed to a third inlet 151 to the additional oligomerization reactor 150. The third inlet 151 may be provided at a second inlet end 148 of the oligomerization reactor 150. The fourth feed stream of C₅ olefins in the fourth feed conduit 171 may also be fed to the third inlet 151 to the additional oligomerization reactor 150. The effluent stream and the fourth feed stream may be fed to the additional oligomerization reactor 150 together through the effluent line 146 to the third inlet 151 or in separate conduits or through separate inlets. The effluent stream may be heat exchanged to adjust its temperature before entering the oligomerization reactor 150. The third inlet 151 is downstream of the first inlet 141 and the second inlet 143 relative to feed flow through the oligomerization zone 130. The fourth feed stream of C₅ olefins in fourth feed conduit 171 may serve as a quench for the effluent from the reactor 138 and/or from bed 144 to avoid excessive temperature rise. A cooler may be on the second pentene line 170 (not shown) or the fourth feed conduit 171 to facilitate quenching, particularly if the fourth feed conduit feeds the reactor 150 separately from effluent line 146.

The fifth feed stream of C₅ olefins in the fifth feed conduit 173 is fed to a fourth inlet 153 to the oligomerization reactor 168. The fourth inlet may be arranged to provide feed to an additional bed 154 or to an interbed location between beds 152 and the additional bed 154. However, the fourth inlet 153 is downstream of the third inlet 151 relative to feed flow through the additional oligomerization reactor 150 and the oligomerization reactor 138 and downstream of the first inlet 141 and the second inlet 143 of the oligomerization reactor 138 relative to feed flow through the oligomerization zone 130. The fifth stream of C₅ olefins may serve as a quench for the effluent from the first bed 152 to avoid excessive temperature rise. A cooler may be on the fifth feed conduit 173 to facilitate temperature adjustment.

Additional oligomerization occurs across bed 154 with an emphasis on nonene production due to the stoichiometric excess of C₄ olefins over C₅ olefins at each feed inlet. Oligomerized product, in an oligomerate stream, exits the first oligomerization reactor 150 in an oligomerate conduit 156. The oligomerate conduit 156 exits the additional oligomerization reactor 150 at an outlet end 158 of the additional oligomerization reactor 150 and the oligomerization zone 130.

Remaining C₄ olefins in the effluent stream oligomerize over the oligomerization catalyst to provide an oligomerate comprising C₄ olefin dimers and trimers. Remaining C₅ olefins, if present in the oligomerization feed stream, oligomerize over the oligomerization catalyst to provide an oligomerate comprising C₅ olefin dimers and trimers and co-oligomerize with C₄ olefins to make C₉ olefins. Over 90 wt % of the C₄ olefins in the first feed stream can oligomerize in the oligomerization reactor zone 130. Over 90 wt % of the C₅ olefins in the each C₅ olefin feed stream 167, 169, 171 and 173 can oligomerize in the oligomerization zone 130. If more than one oligomerization reactor is used, conversion of the C₄ olefins is achieved over all of the oligomerization reactors 138, 150 in the oligomerization zone 130.

A stoichiometric surplus of C₄ olefins to C₅ olefins should be maintained in the feed to the first bed 152 and to the additional bed 154 to promote co-oligomerization of C₄ olefins with C₅ olefins to form nonene oligomers. The fourth feed stream of C₅ olefins in the fourth feed line 171 and the fifth feed stream of C₅ olefins in the fifth feed line 173 should have smaller mass and molar flow rates than the effluent feed stream in the effluent line 146. For example, the weight ratio of C₄ olefins to C₅ olefins in the reactor should be between about 1.5 and about 3.0 and preferably between about 1.7 and about 2.5 at a feed inlet 151, 153 through which a C₅ olefin feed stream is added to the oligomerization reactor 138. Consequently, nonene production is maximized due to the stoichiometric excess of C₄ olefins over C₅ olefins at the feed inlets in the oligomerization reactor 150. The mass flow rate of C₅ olefins to an inlet may have to be reduced for downstream feed streams to account for depletion of C₄ olefins across the oligomerization reactor 138, 150.

The oligomerate conduit 156, in communication with the oligomerization zone 130, withdraws an oligomerate stream from the oligomerization zone 130. The oligomerate conduit 156 may be in downstream communication with the first oligomerization reactor 138 and the additional oligomerization reactor 150.

The oligomerization zone 130 may contain an oligomerization catalyst. The oligomerization catalyst may comprise a zeolitic catalyst. The zeolite may comprise between 5 and 95 wt % of the catalyst. Suitable zeolites include zeolites having a structure from one of the following classes: MFI, MEL, SFV, SVR, ITH, IMF, TUN, FER, EUO, BEA, FAU, BPH, MEI, MSE, MWW, UZM-8, MOR, OFF, MTW, TON, MTT, AFO, ATO, and AEL. These three letter codes for structure types are assigned and maintained by the International Zeolite Association Structure Commission in the ATLAS OF ZEOLITE FRAMEWORK TYPES, which is at http://www.iza-structure.org/databases/. In a preferred aspect, the first oligomerization catalyst may comprise a zeolite with a framework having a ten-ring pore structure. Examples of suitable zeolites having a ten-ring pore structure include those comprising TON, MTT, MFI, MEL, AFO, AEL, EUO and FER. In a further preferred aspect, the oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include MTT. In a further aspect, the oligomerization catalyst comprises an MTT zeolite.

The oligomerization catalyst may be formed by combining the zeolite with a binder, and then forming the catalyst into pellets. The pellets may optionally be treated with a phosphoric reagent to create a zeolite having a phosphorous component between 0.5 and 15 wt % of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the catalyst binder utilized in the present invention is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark Versal. A preferred alumina is available from Sasol North America Alumina Product Group under the trademark Catapal. This material is an extremely high purity alpha-alumina monohydrate (pseudo-boehmite) which after calcination at a high temperature has been shown to yield a high purity gamma-alumina.

A suitable oligomerization catalyst is prepared by mixing proportionate volumes of zeolite and alumina to achieve the desired zeolite-to-alumina ratio. In an embodiment, about 5 to about 80, typically about 10 to about 60, suitably about 15 to about 40 and preferably about 20 to about 30 wt % MTT zeolite and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize surface acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

In an embodiment, the oligomerization catalyst may be a solid phosphoric acid catalyst (SPA). The SPA catalyst refers to a solid catalyst that contains as a principal ingredient an acid of phosphorous such as ortho-, pyro- or tetraphosphoric acid. SPA catalyst is normally formed by mixing the acid of phosphorous with a siliceous solid carrier to form a wet paste. This paste may be calcined and then crushed to yield catalyst particles or the paste may be extruded or pelleted prior to calcining to produce more uniform catalyst particles. The carrier is preferably a naturally occurring porous silica-containing material such as kieselguhr, kaolin, infusorial earth and diatomaceous earth. A minor amount of various additives such as mineral talc, fuller's earth and iron compounds including iron oxide may be added to the carrier to increase its strength and hardness. The combination of the carrier and the additives preferably comprises about 15 to 30 wt % of the catalyst, with the remainder being the phosphoric acid. The additive may comprise about 3 to 20 wt % of the total carrier material. Variations from this composition such as a lower phosphoric acid content are possible. Further details as to the composition and production of SPA catalysts may be obtained from U.S. Pat. No. 3,050,472, U.S. Pat. No. 3,050,473 and U.S. Pat. No. 3,132,109 and from other references. Feed to the oligomerization zone 130 containing SPA catalyst as the oligomerization catalyst should be kept dry except in an initial start-up phase.

The oligomerization reaction conditions in the oligomerization reactors 138, 150 in the oligomerization zone 130 are set to keep the reactant fluids in the liquid phase. With liquid oligomerate recycle, lower pressures are necessary to maintain liquid phase. Operating pressures include between about 2.1 MPa (300 psia) and about 10.5 MPa (1520 psia), suitably at a pressure between about 2.1 MPa (300 psia) and about 6.9 MPa (1000 psia) and preferably at a pressure between about 2.8 MPa (400 psia) and about 4.1 MPa (600 psia). Lower pressures may be suitable if the reaction is kept in the liquid phase.

For the zeolite catalyst, the temperature of the oligomerization zone 130 expressed in terms of a maximum bed temperature is in a range between about 150° and about 300° C. The maximum bed temperature should between about 200° and about 250° C. and preferably between about 215° or about 225° C. and about 245° C. The weight hourly space velocity should be between about 0.5 and about 5 hr⁻¹.

For the SPA catalyst, the temperature in the oligomerization zone 130 should be in a range between about 100° and about 250° C. and suitably between about 150° and about 200° C. The weight hourly space velocity should be between about 0.5 and about 5 hr⁻¹.

Across a single bed of oligomerization catalyst, the exothermic reaction will cause the temperature to rise. Consequently, the oligomerization reactor may be operated to allow the temperature at the outlet to be over about 25° C. greater than the temperature at the inlet.

The oligomerization zone 130 with the oligomerization catalyst can be run in high conversion mode of greater than 95% conversion of feed olefins to produce a high quality diesel product and gasoline product. Normal butene conversion can exceed about 80%. Additionally, normal pentene conversion can exceed about 80%.

We have found that when C₅ olefins are present in the oligomerization feed stream, they dimerize or co-dimerize with other olefins, but tend to mitigate further oligomerization over the zeolite with a 10-ring uni-dimensional pore structure. Best mitigation of further oligomerization occurs when the C₅ olefins comprise between about 15 and about 50 wt % and preferably between about 20 and about 40 wt % of the olefins in the oligomerization feed. Consequently, the oligomerate stream in oligomerate conduit 156 may comprise less than about 60 wt % C₁₂+ hydrocarbons when C₅ olefins are present in the oligomerization feed at these proportions. Furthermore, the net gasoline yield may be at least about 40 wt % when C₅ olefins are present in the oligomerization feed.

An oligomerization recovery zone 200 is in downstream communication with the oligomerization zone 130 and the oligomerate conduit 156. The oligomerate conduit 156 removes the oligomerate stream from the oligomerization zone 130.

The oligomerization recovery zone 200 may include a second debutanizer column 210 which separates the oligomerate stream between vapor and liquid into a first vaporous oligomerate overhead light stream comprising C₄ olefins and hydrocarbons in a first overhead line 212 and a first liquid oligomerate bottom stream comprising C₅+ olefins and hydrocarbons in a first bottom line 214. Maximum production of distillate is desired to recrack the diesel in the FCC zone 20 to make more propylene, the overhead pressure in the debutanizer column 210 may be between about 300 and about 700 kPa (gauge) and the bottom temperature may be between about 225° and about 300° C. The first vaporous oligomerate overhead light stream comprising C₄ hydrocarbons may be rejected from the process and subjected to further processing to recover useful components.

It is desired to maintain liquid phase in the oligomerization reactors. This is typically achieved by saturating product olefins and recycling them to the oligomerization reactor as a liquid. However, if olefinic product is being recycled to either the FCC zone 20 or the oligomerization zone 130, saturating olefins would inactivate the recycle feed. The oligomerization zone 130 can only further oligomerize olefinic recycle and the FCC zone 20 prefers olefinic feed to be further cracked to form propylene.

Liquid phase may be maintained in the oligomerization zone 130 by incorporating into the feed a C₅ stream from the oligomerization recovery zone 200. The oligomerization recovery zone 200 may include a depentanizer column 220 to which the first liquid oligomerate bottom stream comprising C₅+ hydrocarbons may be fed in line 214. The depentanizer column 220 may separate the first liquid oligomerate bottom stream between vapor and liquid into an intermediate stream comprising C₅ olefins and hydrocarbons in an intermediate line 222 and a liquid oligomerate bottom product stream comprising C₆+ olefins in a bottom product line 224. When maximum production of distillate is desired to recrack the diesel in the FCC zone 20 to make more propylene, the overhead pressure in the depentanizer column 220 may be between about 50 and about 100 kPa (gauge) and the bottom temperature may be between about 200° and about 275° C. In the oligomerization recovery zone 200, and specifically in the depentanizer column 220, the oligomerate stream in line 156 is separated into a liquid oligomerate stream comprising C₆+ olefins with a large fraction of C₉ olefins in bottoms product line 224 and an intermediate stream comprising C₅ hydrocarbons in the intermediate overhead line 222.

The intermediate stream in intermediate line 222 may comprise at least 70 wt % and suitably at least 90 wt % C₅ hydrocarbons which can then act as a solvent in the oligomerization zone 130 to maintain liquid phase therein. The overhead intermediate stream comprising C₅ hydrocarbons may have less than 10 wt % C₄ or C₆ hydrocarbons and may preferably have less than 1 wt % C₄ or C₆ hydrocarbons. However, it is also contemplated that the split in the depentanizer column be adjusted, so the overhead stream would have relatively more heavier hydrocarbons.

The intermediate stream may be condensed and recycled to the oligomerization zone 130 as an intermediate recycle stream in an intermediate recycle line 226 to maintain the liquid phase in the oligomerization reactors 138, 150 operating in the oligomerization zone 130. Specifically the intermediate recycle stream in intermediate recycle line 226 comprising C₅ hydrocarbons may be recycled to the oligomerization zone 130 and particularly to the oligomerization reactor 138 through the first inlet 141. The intermediate recycle stream in intermediate recycle line 226 may be combined with the first feed stream before entering the oligomerization reactor 138 comprising C₄ olefins in the first feed conduit 132. The intermediate recycle stream may instead be recycled to the oligomerization reactor 138 separately from the first feed conduit such as with second feed stream in line 167, the third feed stream in third feed line 169, the fourth feed stream in fourth feed line 171 and/or the fifth feed stream in fifth feed line 173. The overhead intermediate stream may comprise a small quantity of unreacted C₅ olefins that can oligomerize when recycled to the oligomerization zone. The C₅ hydrocarbon presence in the oligomerization zone maintains the oligomerization reactors at liquid phase conditions. The pentanes are easily separated from the heavier olefinic product such as in the depentanizer column 220. The pentane recycled to the oligomerization zone also dilutes the feed olefins to help limit the temperature rise within the reactor due to the exothermicity of the reaction.

We have found that dimethyl sulfide boils with the C₅ hydrocarbons and deactivates the unidimensional, 10-ring pore structured zeolite which may be the oligomerization catalyst. The mercaptan extraction unit 112 may not remove sufficient dimethyl sulfide to avoid deactivating the oligomerization catalyst. Consequently, recycle of C₅ hydrocarbons to the oligomerization zone 130 with oligomerization catalyst comprising a unidimensional, 10-ring pore structured zeolite may be avoided by keeping valve 226′ shut unless dimethyl sulfide can be successfully removed from the oligomerate stream or the oligomerization catalyst is not a unidimensional, 10-ring pore structured zeolite. However, the dimethyl sulfide does not substantially harm the solid phosphoric acid catalyst, so recycle of C₅ hydrocarbons to oligomerization zone 130 with such catalysts is suitable.

In an aspect, the intermediate stream in the intermediate line 222 comprising C₅ hydrocarbons may be split into a purge stream in purge line 228 and the intermediate recycle stream comprising C₅ hydrocarbons in the intermediate recycle line 226. In an aspect, the intermediate recycle stream in intermediate recycle line 226 taken from the intermediate stream in intermediate line 222 is recycled to the oligomerization zone 130 downstream of the selective hydrogenation reactor 116. The intermediate recycle stream in intermediate recycle line 226 should be understood to be a condensed overhead stream. The intermediate recycle stream comprising C₅ hydrocarbons may be recycled to the oligomerization zone 130 at a mass flow rate which is at least as great as and suitably no greater than three times the mass flow rate of the oligomerization feed stream in the oligomerization feed line 128 fed to the oligomerization zone 130. The recycle rate may be adjusted by adjusting the control valve 226′ as necessary to maintain liquid phase in the oligomerization reactors and to control temperature rise, and to maximize selectivity to gasoline range oligomer products.

The purge stream comprising C₅ hydrocarbons taken from the intermediate stream may be purged from the process in line 228 to avoid C₅ paraffin build up in the process. The purge stream comprising C₅ hydrocarbons in line 228 may be subjected to further processing to recover useful components or be blended in the gasoline pool.

Two streams may be taken from the liquid oligomerate bottom product stream in bottom product line 224. The FCC recycle stream comprising C₆+ olefins and particularly a high proportion of nonenes in an FCC recycle line 280 may be taken from the liquid oligomerate bottom product stream in bottom product line 224. Flow through FCC recycle line 280 can be regulated by control valve 280′. Accordingly, a liquid product oligomerate stream in bottom product line 224 may be separated from the oligomerate stream in oligomerate line 180. At least a portion of the liquid oligomerate stream having a high proportion of nonenes may be forwarded in line 280 to be cracked to propylene in the FCC unit 20. In another aspect, oligomerate product can be recovered in oligomerate product line 230 regulated by control valve 230′ and sent for further recovery or motor fuel blending.

To make additional propylene in the FCC unit, the FCC recycle line 280 will carry the FCC recycle oligomerate stream as feed to the FCC zone 20. In an aspect, the FCC recycle line 280 is in upstream communication with the FCC reaction zone 20 to recycle oligomerate for fluid catalytic cracking down to propylene or other light olefins. If the FCC zone 20 comprises a single reactor riser 26, the first reactor riser 26 may be in downstream communication with the hydrocarbon feed line 24 and the FCC recycle line 280. If the FCC zone 20 comprises the first reactor riser 26 and a second reactor riser 74, the first reactor riser 26 may be in downstream communication with the hydrocarbon feed line 24 and the second reactor riser 74 may be in downstream communication with the FCC recycle line 280. Hence, in an aspect, the FCC reaction zone 20 is in upstream and downstream communication with oligomerization zone 130, the oligomerization recovery zone 200 and/or FCC recovery zone 100.

We have found that C₆+ oligomerate subjected to FCC processing is converted to light olefins best over a blend of medium or smaller pore zeolite blended with a large pore zeolite such as Y zeolite as explained previously with respect to the FCC zone 20. Additionally, oligomerate produced over the oligomerization catalyst in the oligomerization zone 130 provides an excellent feed comprising a high proportion of C₉ olefins to the FCC zone that can be cracked to yield greater quantities of propylene.

The invention will now be further illustrated by the following non-limiting examples.

### EXAMPLES

### Example 1

Feed 1 in Table 1 was contacted with four catalysts to determine their effectiveness in oligomerizing butenes.

TABLE 1

| Component | Fraction, wt % |
|---|---|
| Propylene | 0.1 |
| Iso-C₄'s | 70.04 |
| isobutylene | 7.7 |
| 1-butene | 5.7 |
| 2-butene (cis and trans) | 16.28 |
| 3-methyl-1-butene | 0.16 |
| acetone | 0.02 |
| Total | 100 |

Catalyst A is an MTT catalyst purchased from Zeolyst having a product code Z2K019E and extruded with alumina to be 25 wt % zeolite. Of MTT zeolite powder, 53.7 grams was combined with 2.0 grams Methocel and 208.3 grams Catapal B boehmite. These powders were mixed in a muller before a mixture of 18.2 g HNO₃ and 133 grams distilled water was added to the powders. The composition was blended thoroughly in the muller to effect an extrudable dough of about 52% LOI. The dough then was extruded through a die plate to form cylindrical extrudates having a diameter of about 3.18 mm. The extrudates then were air dried, and calcined at a temperature of about 550° C. The MTT catalyst was not selectivated to neutralize surface acid sites such as with an amine.

Catalyst B is a SPA catalyst commercially available from UOP LLC.

Catalyst C is an MTW catalyst with a silica-to-alumina ratio of 36:1. Of MTW zeolite powder made in accordance with the teaching of U.S. Pat. No. 7,525,008, 26.4 grams was combined with and 135.1 grams Versal 251 boehmite. These powders were mixed in a muller before a mixture of 15.2 grams of nitric acid and 65 grams of distilled water were added to the powders. The composition was blended thoroughly in the muller to effect an extrudable dough of about 48% LOI. The dough then was extruded through a die plate to form cylindrical extrudates having a diameter of about 1/32″. The extrudates then were air dried and calcined at a temperature of about 550° C.

Catalyst D is an MFI catalyst purchased from Zeolyst having a product code of CBV-8014 having a silica-to-alumina ratio of 80:1 and extruded with alumina at 25 wt % zeolite. Of MFI-80 zeolite powder, 53.8 grams was combined with 205.5 grams Catapal B boehmite and 2 grams of Methocel. These powders were mixed in a muller before a mixture of 12.1 grams nitric acid and 115.7 grams distilled water were added to the powders. The composition was blended thoroughly in the muller, then an additional 40 grams of water was added to effect an extrudable dough of about 53% LOI. The dough then was extruded through a die plate to form cylindrical extrudates having a diameter of about 3.18 mm. The extrudates then were air dried, and calcined at a temperature of about 550° C.

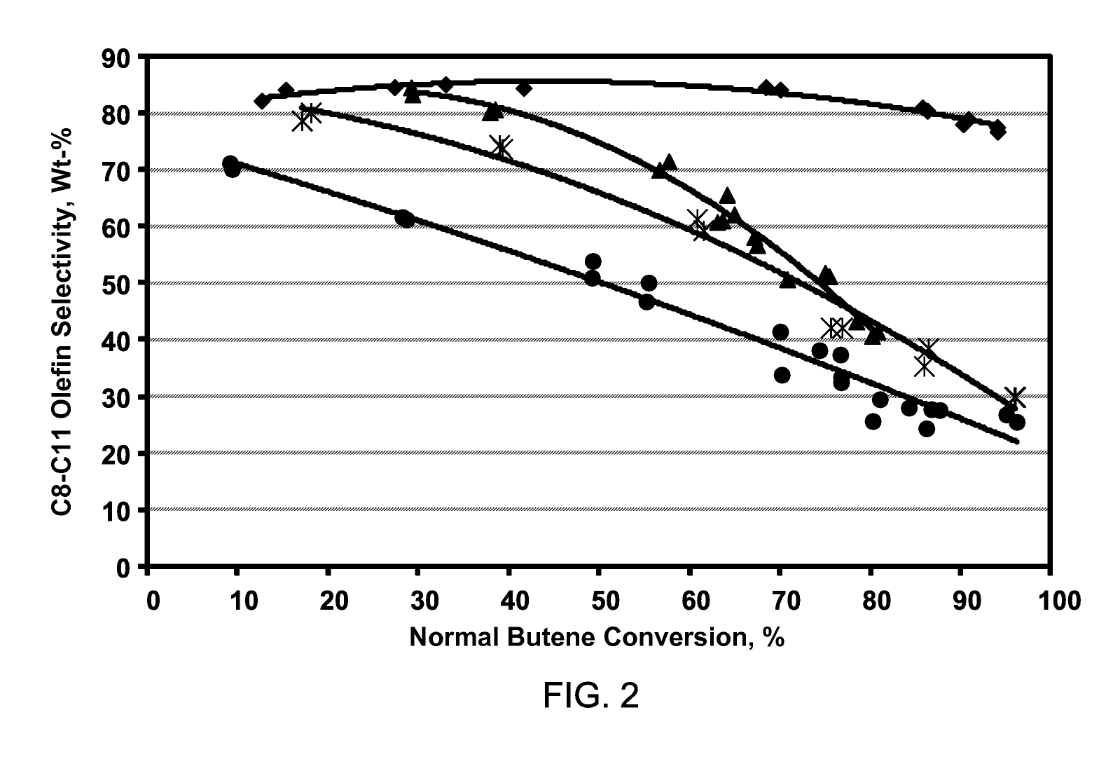

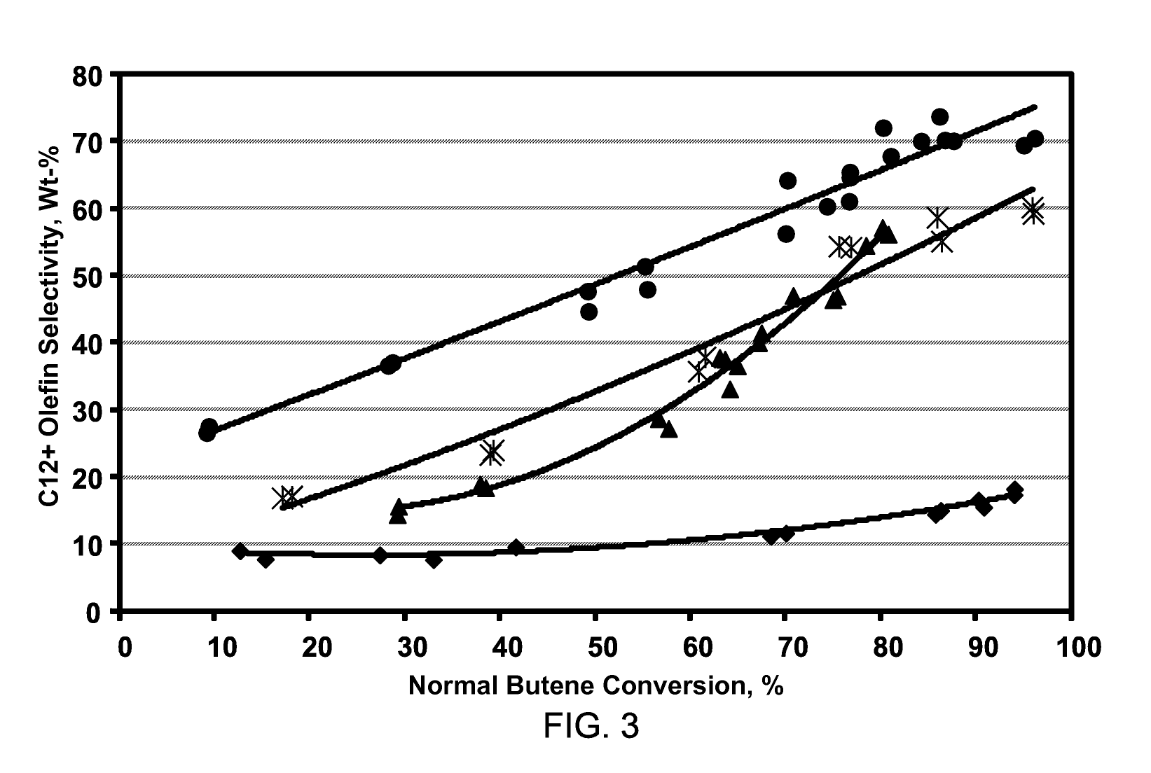

The experiments were operated at 6.2 MPa and inlet temperatures at intervals between 160° and 240° C. to obtain different normal butene conversions. Results are shown in FIGS. 2 and 3. In FIG. 2, C₈ to C₁₁ olefin selectivity is plotted against normal butene conversion to provide profiles for each catalyst.

Table 2 compares the RONC ±3 for each product by catalyst and provides a key to FIG. 2. The RONC was determined for the composite product for each catalyst run per ASTM D2699. The SPA catalyst B is superior for selectivity to gasoline-range olefins, but the MTT catalyst A is the least effective in producing gasoline range olefins.

TABLE 2

| Catalyst | | | RONC |
|---|---|---|---|
| A | MTT | circles | 92 |
| B | SPA | diamonds | 96 |
| C | MTW | triangles | 97 |
| D | MFI-80 | asterisks | 95 |

The SPA catalyst was able to achieve over 95 wt % yield of gasoline having a RONC of >95 and with an Engler T90 value of 185° C. for the entire product. The T-90 gasoline specification is less than 193° C.

In FIG. 3, C₁₂+ olefin selectivity is plotted against normal butene conversion to provide profiles for each catalyst. Table 3 compares the derived cetane number ±2 for each product by catalyst and provides a key to FIG. 3. The cetane number was determined for the composite product for each catalyst run per ASTM D6890.

TABLE 3

| Catalyst | | | Cetane |
|---|---|---|---|
| A | MTT | circles | 41 |
| B | SPA | diamonds | <14 |
| C | MTW | triangles | 28 |
| D | MFI-80 | asterisks | 36 |

FIG. 3 shows that the MTT catalyst provides the highest C₁₂+ olefin selectivity which reaches over 70 wt %. These selectivities are from a single pass of the feed stream through the oligomerization reactor. Additionally, the MTT catalyst provided C₁₂+ oligomerate with the highest derived cetane. Cetane was derived using ASTM D6890 on the C₁₂+ fraction at the 204° C. (400° F.) cut point. Conversely to gasoline selectivity, the MTT catalyst A is superior in producing diesel range olefins, and the SPA catalyst B is the least effective in producing diesel range olefins.

The MTT catalyst was able to produce diesel with a cetane rating of greater than 40. The diesel cloud point was determined by ASTM D2500 to be −66° C. and the T90 was 319° C. using ASTM D86 Method. The T90 specification for diesel in the United States is between 282 and 338° C., so the diesel product meets the U.S. diesel standard.

### Example 2

Two types of feed were oligomerized over oligomerization catalyst A of Example 1, MTT zeolite. Feeds 1 and 2 contacted with catalyst A are shown in Table 4. Feed 1 is from Example 1.

TABLE 4

| | Feed 1 | Feed 2 |
|---|---|---|
| Component | Fraction, wt % | Fraction, wt % |
| propylene | 0.1 | 0.1 |
| isobutane | 70.04 | 9.73 |
| isobutylene | 7.7 | 6.3 |
| 1-butene | 5.7 | 4.9 |
| 2-methyl-2-butene | 0 | 9.0 |
| 2-butene (cis & trans) | 16.28 | 9.8 |
| 3-met-1-butene | 0.16 | 0.16 |
| n-hexane | 0 | 60 |
| acetone | 0.02 | 0.01 |
| Total | 100 | 100 |

In Feed 2, C₅ olefin is made up of 2-methyl-2-butene and 3-methyl-1-butene which comprises 9.16 wt % of the reaction mixture representing about a third of the olefins in the feed. 3-methyl-1-butene is present in both feeds in small amounts. Propylene was present at less than 0.1 wt % in both feeds.

The reaction conditions were 6.2 MPa and a 1.5 WHSV. The maximum catalyst bed temperature was 220° C. Oligomerization achievements are shown in Table 5.

TABLE 5

| | Feed 1 | Feed 2 |
|---|---|---|
| Inlet Temperature, ° C. | 192 | 198 |
| C₄ olefin conversion, % | 98 | 99 |
| nC₄ olefin conversion, % | 97 | 99 |
| C₅ olefin conversion, % | n/a | 95 |
| C₅-C₇ selectivity, wt % | 3 | 5 |
| C₈-C₁₁ selectivity, wt % | 26 | 40 |
| C₁₂-C₁₅ selectivity, wt % | 48 | 40 |
| C₁₆+ selectivity, wt % | 23 | 16 |
| Total C₉+ selectivity, wt % | 78 | 79 |
| Total C₁₂+ selectivity, wt % | 71 | 56 |
| Net gasoline yield, wt % | 35 | 44 |
| Net distillate yield, wt % | 76 | 77 |

Normal C₄ olefin conversion reached 99% with C₅ olefins in Feed 2 and was 97 wt % without C₅ olefins in Feed 1. C₅ olefin conversion reached 95%. With C₅ olefins in Feed 2, it was expected that a greater proportion of heavier, distillate range olefins would be made. However, the Feed 2 with C₅ olefins oligomerized to a greater selectivity of lighter, gasoline range product in the C₅-C₇ and C₈-C₁₁ range and a smaller selectivity to heavier distillate range product in the C₁₂-C₁₅ and C₁₆+ range.

This surprising result indicates that by adding C₅ olefins to the feed, a greater yield of gasoline and nonenes can be made over Catalyst A, MTT. This is confirmed by the greater net yield of gasoline and the lower selectivity to C₁₂+ fraction for Feed 2 than for Feed 1. Also, but not to the same degree, by adding C₅ olefins to the feed a greater yield of distillate range material can be made. This is confirmed by the greater net yield of distillate for Feed 2 than for Feed 1 on a single pass basis. Gasoline yield was classified by product meeting the Engler T90 requirement and distillate yield was classified by product boiling over 150° C. (300° F.).

### Example 3

Three types of feed were oligomerized over oligomerization catalyst B of Example 1, SPA. The feeds contacted with catalyst B are shown in Table 6. Feed 2 is the same as Feed 2 in Example 2. Normal hexane and isooctane were used as heavy paraffin solvents with Feeds 2 and 3, respectively. All feeds had similar C₄ olefin levels and C₄ olefin species distributions. Feed 4 is similar to Feed 2 but has the pentenes evenly split between iso- and normal pentenes, which is roughly expected to be found in an FCC product, and Feed 4 is diluted with isobutane instead of n-hexane

TABLE 6

| | Feed 2 | Feed 3 | Feed 4 |
|---|---|---|---|
| Component | Fraction, wt % | Fraction, wt % | Fraction, wt % |
| propylene | 0.1 | 0.08 | 0.1 |
| 1,3-butadiene | 0 | 0.28 | 0 |
| isobutane | 9.73 | 6.45 | 69.72 |
| isobutylene | 6.3 | 7.30 | 6.3 |
| 1-butene | 4.9 | 5.07 | 4.9 |
| 2-methyl-2-butene | 9.0 | 0 | 4.5 |
| 2-butene (cis & trans) | 9.8 | 11.33 | 9.8 |
| 3-met-1-butene | 0.16 | 0.16 | 0.16 |
| 2-pentene | 0 | 0 | 4.5 |
| cyclopentane | 0 | 0.28 | 0 |
| n-hexane | 60 | 0 | 0 |
| isooctane | 0 | 60.01 | 0 |
| acetone | 0.01 | 0.01 | 0.02 |
| Total | 100 | 100 | 100 |

The reaction pressure was 3.5 MPa. Oligomerization process conditions and testing results are shown in Table 7.

TABLE 7

| | Feed 2 | Feed 3 | Feed 4 |
|---|---|---|---|
| WHSV, hr⁻¹ | .75 | 1.5 | .75 |
| Pressure, MPa | 3.5 | 3.5 | 6.2 |
| Inlet Temperature, ° C. | 190 | 170 | 178 |
| Maximum Temperature, ° C. | 198 | 192 | 198 |
| Total C₄ olefin conversion, % | 95 | 92 | 93 |
| n-butene conversion, % | 95 | 90 | 93 |
| Total C₅ olefin conversion, % | 90 | n/a | 86 |
| C₅-C₇ selectivity, wt % | 8 | 5 | 8 |
| C₈-C₁₁ selectivity, wt % | 77 | 79 | 77 |
| C₁₂-C₁₅ selectivity, wt % | 15 | 16 | 15 |
| C₁₆+ selectivity, wt % | 0.3 | 0.1 | .01 |
| Total C₉+ selectivity, wt % | 35 | 20 | 25 |
| Total C₁₂+ selectivity, wt % | 17 | 16 | 15 |
| Net gasoline yield, wt % | 94 | 92 | 91 |
| Net distillate yield, wt % | 32 | 18 | 23 |
| RONC (±3) | 97 | 96 | 96 |
| Engler T-90, ° C. | 182 | 164 | 182 |

Net gasoline yield goes up to C₁₂− hydrocarbons and net distillate yield goes down to C₉+ hydrocarbons to account for different cut points that may be selected by a refiner. Olefin conversion was at least 90% and normal butene conversion was over 90%. Normal butene conversion reached 95% with C₅ olefins in Feed 2 and was 90% without C₅ olefins in Feed 3. C₅ olefin conversion reached 90% but was less when both iso- and normal C₅ olefins were in Feed 4.

It can be seen that the SPA catalyst minimized the formation of C₁₂+ species to below 20 wt %, specifically, at 16 and 17 wt %, respectively, for feeds containing C₄ olefins or mixtures of C₄ and C₅ olefins in the oligomerization feed stream. When normal C₅ olefins were added, C₁₂+ formation reduced to 15 wt %. The C₆+ oligomerate produced by all three feeds met the gasoline T-90 spec indicating that 90 wt % boiled at temperatures under 193° C. (380° F.). The Research Octane Number for all three products was high, over 95, with and without substantial C₅ olefins present.

### Example 4

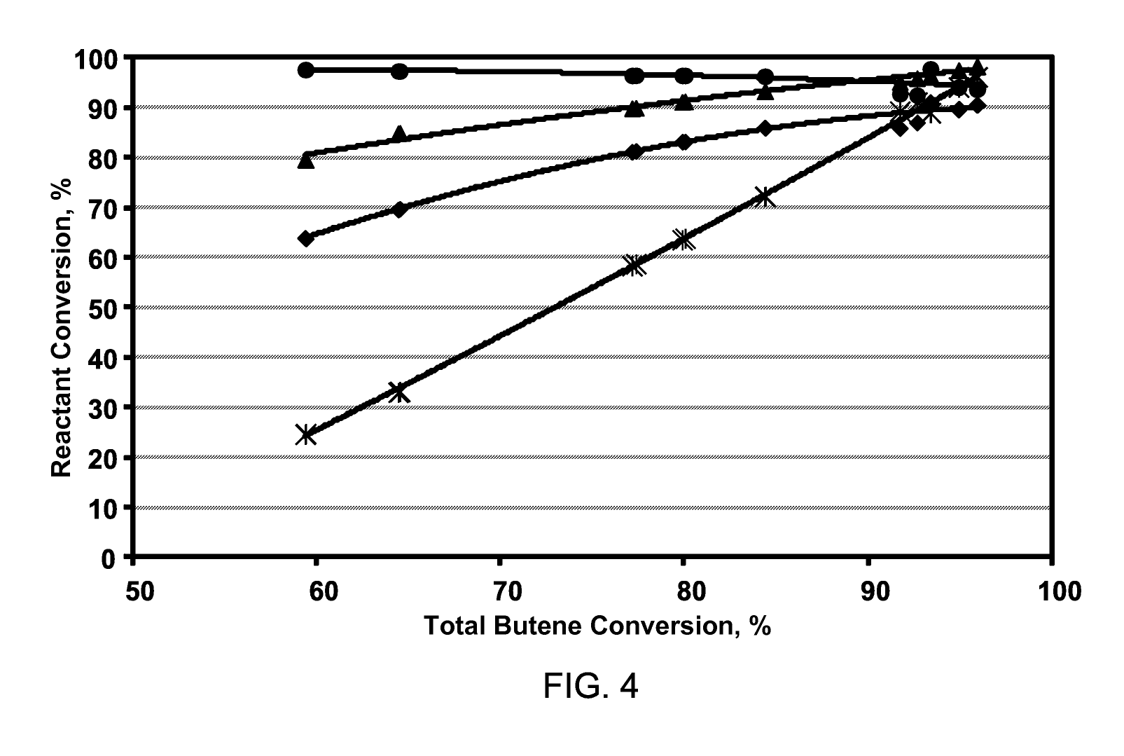

Feed 2 with C₅ olefins present was subjected to oligomerization with Catalyst B, SPA, at different conditions to obtain different butene conversions. C₅ olefin is made up of 2-methyl-2-butene and 3-methyl-1-buene which comprises 9.16 wt % of the reaction mixture representing about a third of the olefins in the feed. Propylene was present at less than 0.1 wt %. Table 8 shows the legend of component olefins illustrated in FIG. 4.

TABLE 8

| Component | Symbols in FIG. 4 |
|---|---|
| isobutylene | Circle |
| 1-butene | Triangle |
| 2-methyl-2-butene and | Diamond |
| 3-met-1-butene | |
| 2-butene (cis & trans) | Asterisk |

FIG. 4 shows conversions for each of the olefins in Feed 2 over Catalyst B, SPA. Over 95% conversion of normal C₄ olefins was achieved at over 90% total butene conversion. Pentene conversion reached 90% at over 90% total butene conversion. Normal butene conversion actually exceeded isobutene conversion at high butene conversion over about 95%.

### Example 5

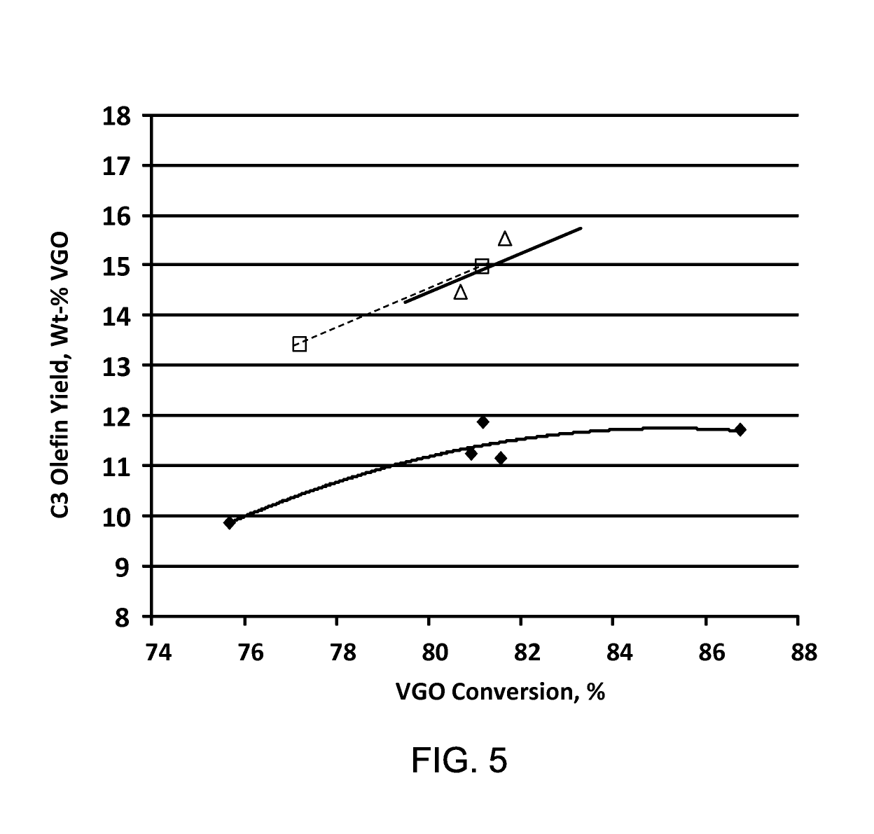

Three feeds were reacted over FCC equilibrium catalyst comprising 8 wt % ZSM-5. Feed 5 comprised hydrotreated VGO with a hydrogen content of 13.0 wt %. Feed 6 comprised the same VGO mixed with 25 wt % oligomerate product catalyzed by Catalyst A of Example 1. Feed 7 comprised the same VGO mixed with 25 wt % oligomerate product catalyzed by Catalyst B of Example 1. The feeds were heated to 260° to 287° C. and contacted with the FCC catalyst in a riser apparatus to achieve 2.5 to 3.0 seconds of residence time. FIG. 5 plots C₃ olefin yield versus VGO conversion. The key for FIG. 5 is in Table 9.

TABLE 9

| Feed | Composition | Key |
|---|---|---|
| Feed 5 | VGO | Solid diamond |
| Feed 6 | VGO/MTT oligomerate | Square |
| Feed 7 | VGO/SPA oligomerate | Triangle |

FIG. 5 shows that recycle of oligomerate product to the FCC zone can boost propylene production. At the apex of the propylene yield curve of VGO alone, the feed comprising VGO and oligomerate provided 3.2 wt % more propylene yield from the FCC zone.

### Specific Embodiments

While the following is described in conjunction with specific embodiments, it will be understood that this description is intended to illustrate and not limit the scope of the preceding description and the appended claims.

A first embodiment of the invention is a process for making olefins comprising feeding a first feed stream comprising C₄ olefins to an oligomerization reactor having an inlet end and an outlet end; feeding a second feed stream comprising C₅ olefins to the oligomerization reactor at a first inlet; feeding a third feed stream comprising C₅ olefins to an oligomerization reactor at a second inlet that is downstream of the first inlet; and oligomerizing the C₄ olefins and the C₅ olefins over an oligomerization catalyst to produce an oligomerate stream comprising C₉ olefins. An embodiment of the invention is one, any or all of prior embodiments in this paragraph up through the first embodiment in this paragraph further comprising providing a C₅ olefin stream and splitting the C₅ olefin stream into the second feed stream and the third feed stream. An embodiment of the invention is one, any or all of prior embodiments in this paragraph up through the first embodiment in this paragraph wherein the second feed stream and the third feed stream have smaller mass flow rates than the first feed stream. An embodiment of the invention is one, any or all of prior embodiments in this paragraph up through the first embodiment in this paragraph further comprising separating a liquid oligomerate stream comprising C₉ olefins from the oligomerate stream. An embodiment of the invention is one, any or all of prior embodiments in this paragraph up through the first embodiment in this paragraph further comprising forwarding the liquid oligomerate stream to a catalytic cracking reactor for conversion to propylene. An embodiment of the invention is one, any or all of prior embodiments in this paragraph up through the first embodiment in this paragraph further comprising separating an intermediate stream comprising C₅ hydrocarbons from the oligomerate stream. An embodiment of the invention is one, any or all of prior embodiments in this paragraph up through the first embodiment in this paragraph further comprising recycling the intermediate stream to the oligomerization reactor. An embodiment of the invention is one, any or all of prior embodiments in this paragraph up through the first embodiment in this paragraph further comprising recycling the intermediate stream to the first feed stream before entering the oligomerization reactor. An embodiment of the invention is one, any or all of prior embodiments in this paragraph up through the first embodiment in this paragraph further comprising separating a purge stream from the intermediate stream and purging the purge stream from the process. An embodiment of the invention is one, any or all of prior embodiments in this paragraph up through the first embodiment in this paragraph further comprising separating a light stream comprising C₄ hydrocarbons from the oligomerate stream.

A second embodiment of the invention is a process for making olefins comprising feeding a first feed stream comprising C₄ olefins to an oligomerization zone having an inlet end and an outlet end; providing a C₅ olefin stream and splitting the C₅ olefin stream into a second feed stream and a third feed stream; feeding the second feed stream comprising C₅ olefins to the oligomerization zone at a first inlet; feeding the third feed stream comprising C₅ olefins to the oligomerization zone at a second inlet that is downstream of the first inlet; and oligomerizing the C₄ olefins and the C₅ olefins over an oligomerization catalyst to produce an oligomerate stream comprising C₉ olefins. An embodiment of the invention is one, any or all of prior embodiments in this paragraph up through the second embodiment in this paragraph wherein the second feed stream and the third feed stream have smaller mass flow rates than the first feed stream. An embodiment of the invention is one, any or all of prior embodiments in this paragraph up through the second embodiment in this paragraph further comprising separating the oligomerate stream into a liquid oligomerate stream comprising C₉ olefins and an intermediate stream comprising C₅ hydrocarbons. An embodiment of the invention is one, any or all of prior embodiments in this paragraph up through the second embodiment in this paragraph further comprising forwarding the liquid oligomerate stream to a catalytic cracking reactor for conversion to propylene. An embodiment of the invention is one, any or all of prior embodiments in this paragraph up through the second embodiment in this paragraph further comprising recycling the intermediate stream to the oligomerization zone.

A third embodiment of the invention is a process for making olefins comprising feeding a first feed stream comprising C₄ olefins to an oligomerization reactor having an inlet end and an outlet end; feeding a second feed stream comprising C₅ olefins to the oligomerization reactor at a first inlet, the second feed stream having a smaller mass flow rate than the first feed stream; feeding a third feed stream comprising C₅ olefins to the oligomerization reactor at a second inlet that is downstream of the first inlet, the third feed stream have smaller mass flow rate than the first feed stream; and oligomerizing the C₄ olefins and the C₅ olefins over an oligomerization catalyst to produce an oligomerate stream comprising C₉ olefins. An embodiment of the invention is one, any or all of prior embodiments in this paragraph up through the third embodiment in this paragraph further comprising separating the oligomerate stream into a liquid oligomerate stream comprising C₉ olefins and an intermediate stream comprising C₅ hydrocarbons. An embodiment of the invention is one, any or all of prior embodiments in this paragraph up through the third embodiment in this paragraph further comprising providing a C₅ stream and splitting the C₅ stream into the second feed stream and the third feed stream. An embodiment of the invention is one, any or all of prior embodiments in this paragraph up through the third embodiment in this paragraph further comprising recycling the intermediate stream to the first feed stream before entering the oligomerization reactor. An embodiment of the invention is one, any or all of prior embodiments in this paragraph up through the third embodiment in this paragraph further comprising forwarding the liquid oligomerate stream to a catalytic cracking reactor for conversion to propylene.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A process for making olefins comprising:
feeding a first feed stream comprising C₄ olefins to an oligomerization reactor having an inlet end and an outlet end;
feeding a second feed stream comprising C₅ olefins to said oligomerization reactor at a first inlet;
feeding a third feed stream comprising C₅ olefins to an oligomerization reactor at a second inlet that is downstream of said first inlet; and
oligomerizing said C₄ olefins and said C₅ olefins over an oligomerization catalyst to produce an oligomerate stream comprising C₉ olefins.

2. The process of claim 1 further comprising providing a stream comprising C₅ olefins and splitting said stream comprising C₅ olefins into said second feed stream and said third feed stream.

3. The process of claim 1 wherein said second feed stream and said third feed stream each have smaller mass flow rates than said first feed stream.

4. The process of claim 1 further comprising separating a liquid oligomerate stream comprising C₉ olefins from said oligomerate stream.

5. The process of claim 4 further comprising forwarding said liquid oligomerate stream to a catalytic cracking reactor for conversion to propylene.

6. The process of claim 4 further comprising separating an intermediate stream comprising C₅ hydrocarbons from said oligomerate stream.

7. The process of claim 6 further comprising recycling said intermediate stream to said oligomerization reactor.

8. The process of claim 7 further comprising recycling said intermediate stream to said first feed stream before entering said oligomerization reactor.

9. The process of claim 1 further comprising separating a purge stream from said intermediate stream and purging said purge stream from said process.

10. The process of claim 1 further comprising separating a light stream comprising C₄ hydrocarbons from said oligomerate stream.

11. A process for making olefins comprising:
feeding a first feed stream comprising C₄ olefins to an oligomerization zone having an inlet end and an outlet end;
providing a stream comprising C₅ olefins and splitting said stream comprising C₅ olefins into a second feed stream and a third feed stream;
feeding said second feed stream comprising C₅ olefins to said oligomerization zone at a first inlet;
feeding said third feed stream comprising C₅ olefins to said oligomerization zone at a second inlet that is downstream of said first inlet; and
oligomerizing said C₄ olefins and said C₅ olefins over an oligomerization catalyst to produce an oligomerate stream comprising C₉ olefins.

12. The process of claim 11 wherein said second feed stream and said third feed stream each have smaller mass flow rates than said first feed stream.

13. The process of claim 11 further comprising separating said oligomerate stream into a liquid oligomerate stream comprising C₉ olefins and an intermediate stream comprising C₅ hydrocarbons.

14. The process of claim 13 further comprising forwarding said liquid oligomerate stream to a catalytic cracking reactor for conversion to propylene.

15. The process of claim 13 further comprising recycling said intermediate stream to said oligomerization zone.

16. A process for making olefins comprising:
feeding a first feed stream comprising C₄ olefins to an oligomerization reactor having an inlet end and an outlet end;
feeding a second feed stream comprising C₅ olefins to said oligomerization reactor at a first inlet, said second feed stream having a smaller mass flow rate than said first feed stream;
feeding a third feed stream comprising C₅ olefins to said oligomerization reactor at a second inlet that is downstream of said first inlet, said third feed stream have smaller mass flow rate than said first feed stream; and
oligomerizing said C₄ olefins and said C₅ olefins over an oligomerization catalyst to produce an oligomerate stream comprising C₉ olefins.

17. The process of claim 16 further comprising separating said oligomerate stream into a liquid oligomerate stream comprising C₉ olefins and an intermediate stream comprising C₅ hydrocarbons.

18. The process of claim 16 further comprising providing a C₅ stream and splitting said C₅ stream into said second feed stream and said third feed stream.

19. The process of claim 16 further comprising recycling said intermediate stream to said first feed stream before entering said oligomerization reactor.

20. The process of claim 16 further comprising forwarding said liquid oligomerate stream to a catalytic cracking reactor for conversion to propylene.
